(19) United States
(12) Patent Application Publication
(10) Pub. No.: US 2008/0183005 A1
(43) Pub. Date: Jul. 31, 2008

(54) Method For Making Alkyl (Meth) Acrylates by Direct Esterification

(75) Inventors: Jean-Michel Paul, Metz (FR); Jacqueline Bessalem, Saint-Avold (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Colombes (FR)

(21) Appl. No.: 11/911,574

(22) PCT Filed: Apr. 6, 2006
(86) PCT No.: PCT/FR06/00758

§ 371 Date: Oct. 15, 2007

(30) Foreign Application Priority Data
Apr. 18, 2005 (FR) 0503831

(51) Int. Cl. C07C 69/54

(52) U.S. Cl. 560/205

(57) ABSTRACT

The invention concerns a improved method for making linear, branched or cyclic C₁-C₁₅ alkyl(meth)acrylates, by direct esterification of (meth)acrylic acid with a corresponding alcohol, said reaction being catalyzed by sulfuric acid. The method includes a step of hydrolyzing the neutralizing aqueous flow followed by extraction of the residual (meth)acrylic acid with solvent consisting entirely or partly of the alcohol used in the reaction. The invention enables organic pollution (COD) released in the final aqueous effluents of the process to be reduced, as well as productivity to be increased.

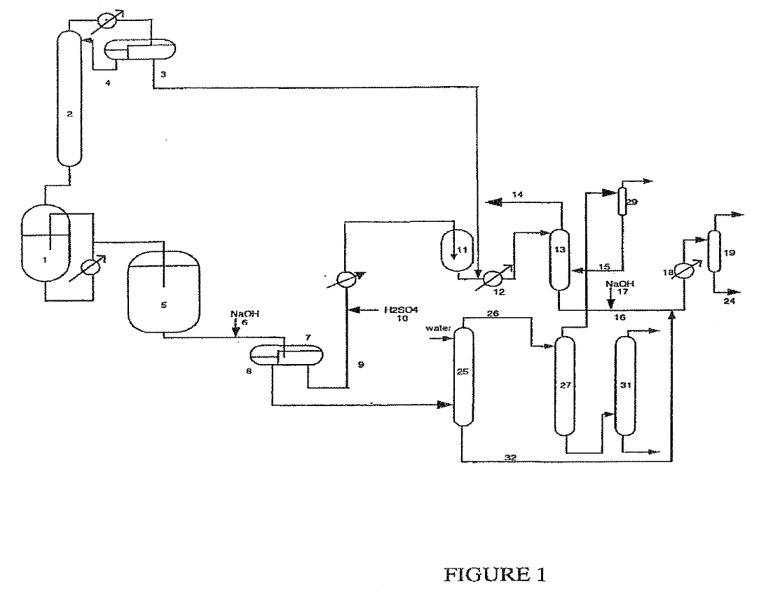

The present invention relates to an improved process for the manufacture of linear, branched or cyclic C₁-C₁₅ alkyl(meth)acrylates by a direct esterification of (meth)acrylic acid by the corresponding alcohol, this reaction being catalyzed by sulfuric acid.

The problems which are posed in the case of this manufacture will now be set out, for convenience, on the basis of the example of the direct esterification of acrylic acid by butanol. These problems and the solution provided by the invention are the same in the case of the use, on the one hand, of methacrylic acid and, on the other hand, of the alcohols other than butanol.

The industrial process for the manufacture of butyl acrylate involves a reaction for the direct esterification of acrylic acid by butanol, a reaction catalyzed by sulfuric acid. In this process, in order to shift the reaction equilibrium, a solvent which azeotropically entrains the water of reaction is not added but this role is provided by an excess of the esterifying alcohol (in this case, butanol), which exhibits the distinguishing feature of forming an azeotrope with water.

On conclusion of the reaction stage, which is carried out batchwise, virtually all of the sulfuric acid has been converted to butyl hydrogen sulfate HBuSO₄, according to the following reaction for the esterification of sulfuric acid by butanol:

BuOH+H₂SO₄→HBuSO₄+H₂O

Consequently, the reaction mixture, at the end of the reaction, comprises butyl acrylate, butanol, acrylic acid, butyl hydrogen sulfate, traces of sulfuric acid and the stabilizers conventionally used in the reaction.

In the conventional process, the esterification reaction is followed by purification stages, generally carried out continuously:
the acid entities present in the crude reaction mixture are neutralized by addition to the latter of an aqueous solution of base (sodium hydroxide); during this stage, the acrylic acid is neutralized to give sodium acrylate, the butyl hydrogen sulfate is neutralized to give neutral butyl sulfate NaBuSO₄ and the traces of sulfuric acid are neutralized to give sodium sulfate Na₂SO₄, all these salts passing into the aqueous phase,
the separated organic phase resulting from the neutralization is washed with water, in an extraction column, in order to remove the traces of impurities, sodium hydroxide and salts and then the washed butyl acrylate is conveyed to a first distillation column, which makes it possible to remove, at the top, the light products which, after topping, are recycled to the esterification reaction. The topped ester is subsequently conveyed to a second column, whence it emerges at the top, purified from the heavy products;
the butanol present is recovered:
   in the separated aqueous phase, resulting from the neutralization, which essentially comprises neutral butyl sulfate, butanol, sodium acrylate and traces of sodium sulfate;
   in the aqueous phase resulting from the esterification reaction proper, and
   in the aqueous phase resulting from the washing of the organic phase in the extraction column,
      this recovery of the butanol being carried out in a distillation column, fed at the top with the aqueous liquors to be treated, in which the butanol is entrained at the top and can be recycled to the esterification reaction, while the column bottom product constitutes the aqueous waste liquors, free from butanol, which will be discharged to the biological treatment plant.

The aqueous liquors thus discharged comprise a high load of organic contamination, measured by the chemical oxygen demand (COD), because of the presence of neutral butyl sulfate, of sodium acrylate and of traces of butyl acrylate and of butanol. An improved process for the manufacture of butyl acrylate which is targeted at reducing the contamination of the aqueous waste liquors has been developed (applicable to other alcohols and methacrylic acid, as indicated above), which formed the subject matter of European patent EP 609 127 of the Applicant Company.

This process comprises an additional stage which consists in hydrolyzing, in an acidic medium and under hot conditions, the butyl hydrogen sulfate in the aqueous phase resulting from the separation carried out after the neutralization stage (where it is present in its saline form), so as to regenerate the butanol and the sulfuric acid:

HBuSO₄+H₂O→BuOH+H₂SO₄

The regenerated butanol is easily removed from the aqueous phase resulting from this treatment, after neutralization, by distillation in the existing column. For this reason, the organic pollution attributable to the butyl hydrogen sulfate (more precisely to the neutral butyl sulfate after neutralization) is greatly reduced.

However, a constituent which is a major contributor to the COD, namely acrylic acid, in the sodium salt form, remains present in the final aqueous effluent. It essentially originates from the residual acrylic acid present in the crude reaction product.

The theoretical COD due to the presence of residual acrylic acid at the end of the reaction is approximately 8 kg of O₂ per ton of butyl acrylate manufactured, which represents more than 60% of the COD discharged in the effluents. In the context of increasing the butyl acrylate production capacity and of increasingly strict legal restrictions with regard to organic discharges, it appears necessary to maintain the COD at an acceptable value by limiting the content of acrylic acid in the final aqueous discharge.

One solution is to minimize the content of residual acrylic acid in the crude reaction product, failing which this is found in the form of sodium acrylate in the final aqueous discharge. Patent application FR 2 228 761 provides a process for the separation of acrylic acid from aqueous solutions by extraction with organic solvents composed of mixtures comprising from 1 to 50 parts by weight of butanol and from 1 to 10 parts by weight of butyl acrylate. Applied to a process for the synthesis of alkyl(meth)acrylates by esterification catalyzed with sulfuric acid, this process generates a large amount of salts and does not make it possible to solve the problem of removal of the alkyl hydrogen sulfate responsible for a large part of the organic contamination emitted in the final aqueous effluent.

U.S. Pat. No. 3,882,167 describes a process for the manufacture of (meth)acrylic esters by esterification on an acidic ion-exchange resin, in which process the residual acrylic acid present in the crude reaction product is neutralized and then, after separation by settling, the aqueous phase is acidified. The acrylic acid present in this phase is subsequently extracted by the distillate from the topping column for the organic phase. This process exhibits the disadvantage of constituting a loop of increasing concentration in light impurities, in particular in dialkyl ether, which risks being re-encountered in the final product. Furthermore, this extraction process, applied to the crude reaction product in a process catalyzed with sulfuric acid, does not make it possible to obtain a satisfactory effectiveness in extracting the alkyl hydrogen sulfate also present in the crude reaction product, which would be largely re-encountered in the aqueous discharge liquors.

Patent application EP 694 524 describes a process for the manufacture of butyl acrylate by direct esterification catalyzed by sulfuric acid which comprises a stage of extraction of the acrylic acid by a solvent chosen from butanol, butyl acrylate or a mixture of butyl acrylate and of butanol. In this process, the crude reaction mixture has pure water or a portion of the water of reaction added to it and is then heated at between 50 and 200° C. in order to hydrolyze the butyl hydrogen sulfate. The hydrolyzed mixture is separated by settling into an acidic organic phase and an acidic aqueous phase. The acidic organic phase is neutralized with sodium hydroxide and then separated by settling. The basic aqueous phase thus separated is brought into contact with the acidic aqueous phase with optionally an addition of sulfuric acid to regenerate the acrylic acid present in the alkali metal salt form. The phase of extraction of the acrylic acid is carried out on this acidic mixture after separation by settling, making it possible to obtain an aqueous phase depleted in organic contamination after distillation of the residual butanol. However, this process exhibits the disadvantage of comprising a first stage of hydrolysis under hot conditions of an organic mixture comprising the reaction product, which can result in its partial hydrolysis to give acrylic acid and butanol and consequently in a fall in yield. Furthermore, numerous stages of separating by settling are carried out, during which interphase problems may arise.

The Applicant Company has thus looked for a novel solution to the problem posed in the process of patent EP 609 127, without this solution exhibiting the abovementioned disadvantages, and it is discovered that the extraction of the acidic aqueous effluent at the outlet of the hydrolysis phase, using a solvent composed in all or part of the alcohol employed in the reaction (in this case, butanol), makes it possible to considerably improve the contamination discharged in the impoverished aqueous liquors from a plant for the manufacture of butyl acrylate: the COD is reduced by at least 40% at the limits of the plant. The purification plant treatment exhibits a cost which is thus found to be reduced. Furthermore, this extraction makes it possible to recover, in the organic phase, the residual acrylic acid present in the crude reaction product and to return it to the reaction stage. The degree of extraction of the acrylic acid is greater than 70% and can be improved by increasing the number of extraction stages.

The principle of the treatment according to the invention, applicable to other alcohols and to methacrylic acid, as indicated above, consists in extracting the acrylic acid present in the acidic aqueous effluent resulting from the hydrolysis phase, which effluent additionally comprises the butanol generated by the hydrolysis of the butyl hydrogen sulfate, sulfuric acid and sodium sulfate. The extraction is carried out with pure butanol or a mixture of butanol and of butyl acrylate, such as, for example, that originating from the topping column of the distillation line. The advantage of using the top stream of the topping column in carrying out the extraction is based essentially on the fact that it is not necessary to introduce pure butanol into the plant, apart from into the reactor, and that the extraction with a stream comprising a small amount of butyl acrylate improves the separation of the water between the organic phase and the aqueous phase, so that the amount of water present in the organic phase to be recycled to the reaction is lower.

The result of this is that the aqueous phase is depleted in acrylic acid and in butanol and can be conveyed to the plant for the treatment of the aqueous effluents, optionally after distillation of the volatile organic products.

The alcoholic organic phase enriched in acrylic acid may or may not be washed with water before returning to the reaction stage, thus without loss of the reaction products.

The technique provided by the invention exhibits the advantage of recovering a large part of the residual acrylic acid resulting from the crude reaction product; it is thus possible to envisage carrying out the reaction at lower degrees of conversion of the acrylic acid without harmful consequences on the discharged COD and, for this reason, reducing the reaction time and increasing the productive output.

A subject matter of the present invention is thus a process for the manufacture of a C₁-C₁₅ alkyl (meth)acrylate by direct esterification of (meth)acrylic acid by the corresponding alcohol, said esterification being catalyzed by sulfuric acid, the crude reaction mixture obtained comprising C₁-C₁₅ alkyl (meth)acrylate, alcohol, (meth)acrylic acid, C₁-C₁₅ alkyl hydrogen sulfate, traces of sulfuric acid and the usual impurities, according to which process the esterification is followed by:
(a) the addition to said crude reaction mixture of a base in order to neutralize the (meth)acrylic acid, the C₁-C₁₅ alkyl hydrogen sulfate and the traces of sulfuric acid which are present therein, the resulting salts passing into the aqueous phase of said mixture, the organic phase and the aqueous phase resulting from this neutralization being separated and the desired alkyl(meth)acrylate being recovered from said organic phase;
(b) the hydrolysis of the alkyl hydrogen sulfate present in said aqueous phase, after acidification of the latter, in order to form alcohol and sulfuric acid,
   characterized in that the (meth)acrylic acid present in said aqueous phase is extracted, after the hydrolysis stage (b), with a solvent composed in all or part of the alcohol employed in the reaction, said aqueous phase, depleted in (meth)acrylic acid and freed from said alcohol, being distilled before being discharged and the extracted organic phase being returned to the reaction stage.

This extraction can be carried out continuously or batchwise.

According to one embodiment, the extraction solvent is the alcohol employed in the esterification reaction.

According to one embodiment, the extraction solvent comprises at least 20% and more particularly at least 40% of alcohol employed in the reaction.

The extraction solvent can additionally comprise the ester resulting from the esterification reaction, water or other byproducts resulting from the reaction.

According to a particularly preferred embodiment, the extraction solvent is composed of the stream originating from the distillate from the first distillation column for the organic phase resulting from the phase separation which follows the first neutralization of the process, which was subsequently purified from the light compounds by topping on another distillation column.

The extraction of the (meth)acrylic acid is carried out on the acidic aqueous effluent resulting from the hydrolysis phase, which effluent additionally comprises the alcohol generated by hydrolysis of the alkyl hydrogen sulfate, sulfuric acid and sodium sulfate. The pH of this aqueous stream is generally between 1 and 3 and more particularly between 1.1 and 1.4.

The extraction of the (meth)acrylic acid is carried out using a device which provides the contact between the acidic aqueous stream comprising the acid to be extracted and the extraction solvent. The devices which can be used for the extraction are, for example: plate columns, packed columns, stirred vessels also acting as decanters, or extraction arrays of Robatel® type. The extraction device generally comprises between 1 and 5 extraction stages, depending on the initial content of (meth)acrylic acid and the degree of recovery desired; a higher number of stages can be used. The effectiveness of the extraction of the (meth)acrylic acid increases as the number of stages increases.

The ratio by weight of the extraction solvent to the stream to be treated is between 0.1/1 and 2.5/1 and more particularly between 1/1 and 2/1.

The solvent and the stream to be treated are brought into contact at a temperature of between 10° C. and 50° C. for a period of time of at least 10 minutes per extraction stage, preferably for 20 to 30 minutes per extraction stage.

The extraction can be carried out for aqueous streams originating from crude reaction products comprising a content by weight of residual (meth)acrylic acid of between 0.1% and 9% and preferably between 0.2% and 5%.

The extracted organic phase, enriched in (meth)acrylic acid, may or may not be washed with water before returning to the esterification stage.

The acidic aqueous effluent depleted in (meth)acrylic acid and in alcohol is subsequently neutralized by a base, for example NaOH, up to a pH of the order of 11-12 and then topped by distillation, in order to remove therefrom the volatile organic products, before being directed to the treatment plant for the aqueous effluents.

According to the process of the invention, the ester formed is recovered by washing with water, in an extraction column, the organic phase resulting from the phase separation which follows the first neutralization, by then sending the washed alkyl (meth)acrylate to a first distillation column, in order to remove the light products at the top, and by finally sending the topped alkyl(meth)acrylate to a second distillation column, whence it emerges at the top purified from the heavy products.

In order to better illustrate the process of the present invention, an embodiment thereof will be described below with reference to the appended drawing, in the case of the manufacture of butyl acrylate.

According to the invention, butyl acrylate (BUA) is prepared by esterification of acrylic acid (AA) by butanol (BuOH), catalyzed by sulfuric acid, in the reactor (1). The water of reaction is continuously removed by distillation (2) in the form of an azeotrope with the butanol. This azeotrope separates on settling into two phases, an organic phase (4), which is returned to the column (2), and an aqueous phase (3), which comprises acrylic acid.

The crude reaction product (5) comprises butyl acrylate, residual acrylic acid, butyl hydrogen sulfate, excess butanol and impurities of the dibutyl ether type or heavy impurities of the Michael type. For a BuOH/AA molar ratio of 1.3/1, the composition by weight of the crude reaction product is, for example, as follows:

| | |
|---|---|
| BUA: | 84% |
| BuOH: | 11% |
| AA: | 0.2% |
| Butyl hydrogen sulfate (HBuSO₄): | 1.8% |
| Impurities: | 3% |

The crude reaction product resulting from the reactor (1) is stored in the vessel (5), then neutralized with an aqueous sodium hydroxide solution (6) at ambient temperature and then separated by settling in (7).

The organic phase (8) is subsequently washed with water in the column (25), in order to free it from the salts dissolved therein, and then directed, via the line (26), to the topping column (27) and the tailing column (31). The butyl acrylate exits at the top of the column (31) purified from the heavy products.

The distillate from the column (27) is topped on the column (29). The bottom product from this column (15), freed from the light compounds of dibutyl ether type, is used in the device (13) as extraction solvent for the acrylic acid present in the effluent resulting from the hydrolyzer (11), to which is optionally added other aqueous effluents which comprise acrylic acid, for example the aqueous effluent (3).

The aqueous phase (9) resulting from the decanter (7) is hydrolyzed in the hydrolyzer (11) in the presence of sulfuric acid (10) as catalyst and neutralization agent for the basic entities present. The amount of sulfuric acid is such that the molar ratio R_{H} of the number of H⁺ equivalents in excess to the number of moles of butyl hydrogen sulfate present in the aqueous phase (9) is at least equal to 1.5. The pH is typically between 1 and 3, preferably between 1.1 and 1.4. The hydrolysis is carried out at a temperature of between approximately 70° C. and 200° C., preferably between 110° C. and 140° C., for a period of time which depends on the temperature applied, the pressure depending on the reaction temperature.

The acidic aqueous effluent at the outlet of the hydrolyzer (11), which comprises the butanol generated by the hydrolysis of the butyl hydrogen sulfate, acrylic acid, sulfuric acid and sodium sulfate, is cooled to a temperature of between 10° C. and 50° C. by virtue of the heat exchanger (12) and then subjected to extraction in (13) with butanol or, preferably, with the stream (15) appearing in the appended diagram composed of the distillate from the column (27) which has subsequently been topped on the column (29). The mean composition of this stream (15) is as follows:

| | |
|---|---|
| BUA: | 25-35% |
| BuOH: | 55-60% |
| Water: | 8-11% |
| Butyl acetate: | 1-5% |

The organic phase (14) enriched in acrylic acid may or may not be washed with water before returning to the reaction (1).

The acidic aqueous effluent depleted in acrylic acid (16) is neutralized with sodium hydroxide (17) to a pH of between 11 and 12 and then conveyed to a distillation column (19), via the exchanger (18), in order to recover, on the one hand, the butanol and the volatile organic products as top product and, on the other hand, the impoverished aqueous liquors intended for the biological treatment as bottom product (24).

The degree of extraction of the acrylic acid according to the invention is greater than 70% and this degree can be improved by increasing the number of extraction stages. A reduction of at least 40 to 50% in the COD of the impoverished aqueous liquors is obtained on conclusion of this process. The process according to the invention additionally exhibits the advantage of simplified implementation with respect to the processes of the state of the art.

Mention may be made, as preferred alcohols for the esterification reaction according to the invention, of methanol, butanol, hexanol and 2-ethylhexanol.

The present invention will now be further illustrated by examples given without implied limitation. In these examples, the percentages are expressed as percentages by weight, unless otherwise indicated, and the following abbreviations have been used:

| | |
|---|---|
| AA: | acrylic acid |
| BUA: | butyl acrylate |
| E2HA: | 2-ethylhexyl acrylate |
| BuOH: | butanol |
| E2HOH: | 2-ethylhexanol |

### EXAMPLE 1

79.7 g of acidic aqueous effluent (pH 1.7) withdrawn at the outlet of the hydrolyzer (11), such as appearing in the appended scheme, for the manufacture of BUA are mixed with stirring for 15 min at ambient temperature with 135.5 g of an effluent stream (15) used as extraction solvent originating from the distillate (27) topped from the light compounds of butyl ether type in the column (29).

The composition by weight of the aqueous effluent is as follows:

| | |
|---|---|
| BuOH: | 2.5% |
| BUA: | 195 ppm |
| AA: | 1.7%, i.e. 1.35 g |
| Na₂SO₄: | 8.5% |

The composition by weight of the extraction solvent is as follows:

| | |
|---|---|
| BUA: | 32% |
| BuOH: | 56% |
| Water: | 85% |
| Butyl acetate: | 3.5% |

The ratio by weight of the solvent to the acidic effluent to be depleted in AA is 1.7/1.

After separation by settling, the following are recovered:
74.2 g of aqueous phase comprising 0.3% of AA, i.e. 0.22 g of AA
135.7 g of organic phase comprising 0.84% of AA, i.e. 1.14 g of AA.

The degree of extraction of the AA by the solvent is thus 84%.

The starting acidic aqueous effluent comprising 1.7% of AA and the acidic aqueous effluent depleted in AA by extraction are neutralized to pH 11-12 and then topped by distillation in order to remove the volatile organic products therefrom. The COD measured after this topping is respectively:
38 700 mg/l for the starting aqueous effluent
14 800 mg/l for the effluent depleted in AA by extraction.

The COD saving is thus 62%.

### EXAMPLE 2

Example 1 is repeated starting from an acidic aqueous effluent of pH 2.3 withdrawn at the outlet of the hydrolyzer (11). The degree of extraction of the AA by the solvent is then 76%.

### EXAMPLE 3

Example 1 is repeated while carrying out the extraction with pure butanol at an acidic aqueous effluent/butanol ratio by weight of 1/1.

The degree of extraction obtained, everything otherwise being equal, with this solvent is 83%.

### EXAMPLE 4

Example 1 is repeated while carrying out the extraction on an aqueous effluent at the outlet of the hydrolyzer (11) comprising 3.5% of AA:
weight of acidic aqueous effluent to be treated: 181.5 g
pH of the effluent: 1.2
weight of solvent: 308.6 g
solvent/effluent ratio by weight=1.7

After mixing for 15 min at ambient temperature, the following are separated by settling:
174.7 g of aqueous phase comprising 0.45% of AA, i.e. 0.78 g of AA
306.8 g of organic phase comprising 1.75% of AA, i.e. 5.37 g of AA

The degree of extraction of the AA by the solvent is 84.5%. With two extraction stages, the degree of extraction is brought to 92.2%.

### EXAMPLE 5

180 g of an acidic aqueous stream originating from the manufacture of E2HA withdrawn at the outlet of the hydrolyzer (11) including 0.94% of AA (pH 1.2) are brought into contact, with stirring at ambient temperature for 15 min, with 306 g of solvent having the following composition:

| | |
|---|---|
| E2HA: | 62.9% |
| E2HOH: | 29.5% |
| 2-ethylhexyl acetate: | 2.5% |
| water: | 1.2% |
| q.s. for 100%: | 3.9% |

After separating by settling, two phases are recovered:
aqueous phase: 172.1 g, comprising 0.3 g of AA
organic phase: 307.9 g, comprising 1.4 g of AA

The degree of extraction of the AA is 82%.

### EXAMPLE 6

Example 5 is repeated starting from an acidic aqueous stream at the outlet of the hydrolyzer (11) which is richer in AA (3.2%). In this case, the degree of extraction obtained in the solvent phase is, everything otherwise being equal, 78%. It exceeds 90% on carrying out the operation over two extraction stages.

### EXAMPLE 7

The stream from the outlet of the hydrolyzer (11) originating from the manufacture of BUA is directed, at a flow rate of 12.8 kg/h, to a Robatel® liquid/liquid extractor composed of three extraction stages.

The overall composition by weight of this stream is as follows:

| | |
|---|---|
| BuOH: | 5.15% |
| BUA: | 0.02% |
| Water: | 84.4% |
| AA: | 4.7% |

The extraction solvent (15) is fed countercurrentwise at a flow rate of 22.6 kg/h. Its composition by weight is as follows:

| | |
|---|---|
| BuOH: | 62.6% |
| BUA: | 25% |
| Water: | 11% |
| Butyl acetate: | 1.8% |
| Butyl ether: | 0.2% |

The residence time in the extractor is 25.6 min and the temperature is 36° C. The solvent/stream to be treated ratio by weight is 1.8.

The organic phase recovered (24.3 kg/h) at the outlet of the third stage has the following composition by weight:

| | |
|---|---|
| BuOH: | 59.7% |
| BUA: | 22% |
| Water: | 15.9% |
| AA: | 2.4% |

The aqueous phase at the extractor outlet (11.3 kg/h) has the following composition by weight:

| | |
|---|---|
| BuOH: | 2.7% |
| BUA: | none |
| AA: | 0.17% |
| Water: | 96.3% |

The degree of extraction of the acrylic acid is 95%.

The COD measured at the inlet of the extractor is 458 g/h.

The COD measured at the outlet of the extractor, in the aqueous extraction phase, is 21 g/h, i.e. a COD saving of 96%.

1. A process for the manufacture of a C₁-C₁₅ alkyl(meth)acrylate by direct esterification of (meth)acrylic acid by a corresponding alcohol, said esterification being catalyzed by sulfuric acid to obtain a crude reaction mixture comprising C₁-C₁₅ alkyl(meth)acrylate, alcohol, (meth)acrylic acid, C₁-C₁₅ alkyl hydrogen sulfate, traces of sulfuric acid and impurities, said direct esterification process followed by:
adding to said crude reaction mixture a base, to form an aqueous phase and an organic phase, and to neutralize the (meth)acrylic acid, the C₁-C₁₅ alkyl hydrogen sulfate and the traces of sulfuric acid present therein to produce salts which pass into the aqueous phase of said mixture;
separating the aqueous phase resulting from this neutralization from said organic phase;
recovering C₁-C₁₅ alkyl(meth)acrylate from said organic phase;
hydrolyzing the alkyl hydrogen sulfate present in said aqueous phase after acidification of said aqueous phase, to form alcohol and sulfuric acid,
characterized in that the C₁-C₁₅ alkyl(meth)acrylic acid present in said aqueous phase is extracted after the hydrolysis with an extraction solvent comprising the corresponding alcohol; thereafter distilling, said aqueous phase, depleted of C₁-C₁₅ alkyl (meth)acrylic acid and freed from said alcohol, before being discharged and returning the extracted organic phase to the esterification step.

2. The process as claimed in claim 1, characterized in that the extraction is carried out continuously.

3. The process as claimed in claim 1, characterized in that the extraction is carried out batchwise.

4. The process as claimed in claim 1, characterized in that the extraction solvent is said corresponding alcohol.

5. The process as claimed in claim 1, characterized in that the extraction solvent comprises the bottoms stream from a second distillation column wherein said second distillation column separates a feed stream comprising the distillate from a first distillation column which separates a feed stream comprising said organic phase after removal of salts via water wash in an extraction column.

6. The process as claimed in claim 1, characterized in that the pH of the stream aqueous phase to be treated is between 1 and 3.

7. The process as claimed in claim 1, characterized in that the ratio by weight of the extraction solvent to the aqueous phase to be treated is between 0.1/1 and 2.5/1.

8. The process as claimed in claim 1, characterized in that the extraction is carried out using a device comprising between 1 and 5 extraction stages.

9. The process as claimed in claim 8, characterized in that the extraction solvent and the aqueous phase to be treated are brought into contact at a temperature of between 10° C. and 50° C. and for a period of time of at least 10 minutes per extraction stage.

10. The process as claimed in claim 1, characterized in that the aqueous phase to be treated originates from a crude reaction product comprising a content by weight of residual (meth)acrylic acid of between 0.1% and 9%.

11. The process as claimed in claim 1, characterized in that the recovery of the C₁-C₁₅ alkyl(meth)acrylate is via washing the organic phase with water, in an extraction column then sending the washed alkyl (meth)acrylate to a first distillation column, in order to remove the light products at the top, and sending the bottoms containing alkyl(meth)acrylate to a third distillation column to provide purified distillate.

12. The process as claimed in claim 1, characterized in that said corresponding alcohol is selected from methanol, butanol, hexanol or 2-ethylhexanol.

13. The process as claimed in claim 1, characterized in that the pH of the aqueous phase to be treated is between 1.1 and 1.4.

14. The process as claimed in claim 1, characterized in that the ratio by weight of the extraction solvent to the aqueous phase to be treated is between 1/1 and 2/1.

15. The process as claimed in claim 8, characterized in that the extraction solvent and the aqueous phase to be treated are brought into contact at a temperature of between 10° C. and 50° C. and for a period of time of at least 20 to 30 minutes per extraction stage.
